(19) United States
(12) Patent Application Publication
(10) Pub. No.: US 2005/0272143 A1
(43) Pub. Date: Dec. 8, 2005

(54) Solid support comprising a functionalized electricity conductor or semiconductor surface, method for preparing same and uses thereof

(76) Inventors: Christophe Bureau, Suresnes (FR); Brigitte Mouanda, Yvelines (FR); Sami Ameur, Tunisienne (FR); Julienne Charlier, Gif-sur-Yvette (FR); Serge Palacin, Bretonneux (FR)

Correspondence Address: ALSTON & BIRD LLP;BANK OF AMERICA PLAZA, 101 SOUTH TRYON STREET, SUITE 4000, CHARLOTTE, NC, 28280-4000, US

(21) Appl. No.: 10/518,923

(22) PCT Filed: Jun. 16, 2003
(86) PCT No.: PCT/FR03/01814

§ 371 Date: Jul. 12, 2005

(30) Foreign Application Priority Data
Jul. 4, 2002 (FR) 02/08381

(51) Int. Cl. C12M 1/34

(52) U.S. Cl. 435/287.2

(57) ABSTRACT

The invention concerns a solid support comprising a functionalized electricity conductor or semiconductor surface coated with a functionalized electrografted organic layer wherein at least 90% of the number of functional groups of interest is accessible. The invention also concerns the method for preparing such a support and uses thereof, in particular as adhesive primer for fixing molecules of interest or objects bearing a complementary function (molecular adhesive).

The present invention relates to a functionalized solid support comprising an electrically conducting or semiconducting surface coated with a functionalized electrografted organic layer within which at least 90% of the number of functional groups is accessible, to the method for preparing such a support, and also to the uses thereof, in particular as an adhesion primer for attaching molecules of interest or objects bearing a complementary function (“molecular Velcro®”).

The functionalization of a surface is the operation by means of which a molecule of interest (for example a molecule having proven properties in solution) is successfully attached to a surface, in such a way—at least—that it conserves thereon all or some of its properties. The functionalization of a surface therefore assumes that the molecule of interest and an associated method for attaching it to the surface are available.

Since the molecule of interest is most commonly an organic (or organometallic) molecule, the method most commonly used consists in calling upon the very large library of organic chemistry reactions: the logic is merely to be able to find functional groups, respectively on the surface and on the molecule of interest, which are compatible, i.e. which can readily—and if possible rapidly—react with one another.

For example, when a surface containing hydroxyl or amine groups is available, it may be functionalized by giving the molecule of interest for example isocyanate or siloxane groups, as is for example described in patent application EP-A-1 110 946, in international application WO 00/51732 or in U.S. Pat. No. 6,258,454, or else acid chlorides as is described in patent application FR-A-2 781 232.

When the molecule of interest does not have functional groups that are directly compatible with those of the surface, this surface may be prefunctionalized with a bifunctional intermediate organic molecule, one of the functional groups of which is compatible with those of the surface, and the other with those of the molecule that it is desired to attach. The molecule is sometimes referred to as an adhesion primer (see, for example: E. P. Plueddmann, in “Fundamentals of Adhesion”, L. H. Lee (Ed.), p. 279, Plenum Press, New York (1990)).

According to the present invention, it is the attachment of this adhesion primer that should be considered as the molecule of interest: the focus here is the manner in which a first organic fragment is attached to a surface, in particular when it is inorganic, the subsequent post-functionalization steps being considered as pure organic reactions.

From this point of view, it is noted that the functionalization of a surface is merely a particular case of organic chemistry reactions, in which one of the two reactants is a surface rather than a molecule in solution. Admittedly, the kinetics associated with heterogeneous reactions between a solution and a surface are substantially different from the analogous reaction in a homogeneous phase, but the reaction mechanisms are, in principle, identical.

In certain cases, the surface is activated by pretreating it so as to create thereon functional groups with higher reactivity, so as to obtain a faster reaction. These may in particular be unstable functional groups, formed transiently, such as for example radicals formed by vigorous oxidation at the surface, either chemically or via irradiation:
it is possible to functionalize a surface bearing nitrogenous groups by bombarding it with particles (ions, electrons, protons, etc.) so as to convert these nitrogenous groups to nitrenes, which can react with a large number of organic functional groups, as has already been described in international application WO 98/22542 and also in U.S. Pat. No. 6,022,597;
it is possible to functionalize a surface by subjecting it to a plasma treatment in which the plasma gas contains a monomer capable of reacting with the reactive groups formed during the irradiation, as has already been described in U.S. Pat. No. 6,287,687 and in international application WO 01/34313;
it is possible to functionalize a hydroxylated surface by strongly oxidizing it with metal salts, so as to produce thereon radicals capable of initiating organic polymerization reactions, as has, for example, already been described in U.S. Pat. Nos. 4,421,569; 5,043,226 and 5,785,791;
it is possible to functionalize a surface by means of radicals, either by irradiation with heavy ions, as described for example in U.S. Pat. No. 6,306,975, or thermally, as described for example in international application WO 98/49206, or else photochemically, as described for example in international application WO 99/16907, etc.

In all these examples, the list of which is not exhaustive, either the surface or the molecule of interest is therefore modified, in such a way that, once modified, the attachment between the two entities amounts to a reaction that is known, moreover, in the library of organic chemistry reactions.

Now, it is observed that this reasoning is only possible insofar as the surface has an electron structure similar to that of an insulator: a physicist might say that the surface must have localized states. A chemist might say that it must have functional groups.

When the surface is a conductor or a semiconductor that is undoped or relatively undoped, such localized states do not exist: the electronic states of the surface are delocalized states. In other words, the notion of a “functional group” (in the organic chemistry sense) has no meaning, and it is thus impossible to use the library of organic chemistry reactions to attach a molecule of interest to a surface.

Two notable exceptions exist: these are the spontaneous chemical reactions of thiol functions (—SH, see in particular: Z. Mekhalif et al., Langmuir, 1997, 13, 2285) and of isonitriles (—N═C, see for example: V. Huc et al., J. of Physical Chemistry B, 1999, 103, 10489) on metal surfaces, and in particular on gold surfaces.

However, these reactions cannot be exploited in all situations. Specifically, thiols, for example, give rise to weak sulfur/metal bonds. These bonds are broken, for example, when the metal subsequently undergoes cathodic or anodic polarization, to form thiolates and sulfonates, respectively, which desorb.

Apart from these two isolated examples, no simple chemical reaction exists for functionalizing electrically conducting or semiconducting surfaces.

The means most commonly used for attaching organic molecules to electrically conducting or semiconducting surfaces is to circumvent the difficulty by equating it to a known problem. It is a matter of forming, on these surfaces, beforehand, hydroxyl groups by ensuring the promotion of an oxide layer (totally or partially hydrated) on the metal. On graphite, which has no solid oxide, anodization nevertheless produces hydroxyl groups which may be exploited (under certain conditions, it is also possible to produce thereon carboxyl groups). When it has been possible to form hydroxyl groups on the surface, this equates to a surface that has localized surface electronic states, i.e. functional groups, and the situation equates to a known problem. In particular, it is then possible to apply all the functionalization processes that have been listed above for insulating surfaces.

However, besides the fact that it is impossible to form an oxide layer on gold or on many noble metals, a large part of the responsibility for the solidity of the interface which will be manufactured between the organic molecule of interest and the metal surface is attributed to the oxide layer and to the method for obtaining it (now, certain oxides, in particular when they are non-stoichiometric, are non-covering, or even non-adhesive). In addition, this route requires at least two or three steps to result in the attachment of a molecule of interest, since the oxide layer must first be constructed before attaching the molecule itself (two steps), or alternatively before attaching an adhesion primer which will allow the attachment of the molecule of interest (three steps).

It is also possible to electrochemically attach organic fragments to conducting or semiconducting surfaces.

The process described in international application WO 98/44172 in fact makes it possible to attach organic functional groups to conducting surfaces. This is a process by which a conducting surface is placed under potential (cathodic) in a solution containing aryl diazonium salts, functionalized with the functional group that it is desired to attach to the surface. Now, the aryl diazonium salts are produced from an aromatic amine, by means of a diazotization reaction using sodium nitrite in hydrochloric medium. This step requires a very low pH, and is therefore not compatible with all the functional groups that it is desired to attach. It is known, for example, that it is impossible to diazotize an aromatic amine bearing a succinimide group (which is useful for attaching a molecule of interest bearing hydroxide or amine groups), or bearing an amine group or else a pyridine group, and that it is difficult for the diazonium functional groups to be compatible with unsaturated bonds, for which they can readily bring about free-radical polymerization.

In certain cases, when no functional group is compatible both with those of the molecule of interest and with the diazotization reaction, the use of the process of grafting diazonium salts thus necessitates the intervention of an intermediate step during which the electrografted layer is functionalized with a bifunctional adhesion primer, at least one of the groups of which is compatible with the functional groups of the molecule of interest.

Furthermore, this process does not make it possible, in practice, to produce thick layers, which leads to a relatively small number of grafted functional groups which are very close to the surface. The functional groups that have been grafted are, overall, moderately accessible for subsequent functionalization reactions with an organic molecule. The most direct practical consequence of this comment is that the post-functionalization reactions on conducting surfaces coated with an organic layer according to this process are slow.

Now, it has been possible, since the 1980s, to electrograft polymers derived from vinyl or cyclic monomers onto electrically conducting and semiconducting surfaces, as is described, for example, in patent application EP-A-0 038 244. This process makes it possible to produce covalent chemical bonds between an organic polymer and an electrically conducting or semiconducting surface. These organic layers constitute, a priori, ideal candidates as a primary layer of attachment of organic or organometallic molecules, since, when a polymer chain is grafted at a point of the surface, a large number of functional groups is grafted per surface site: the number of points of attachment of an organic molecule per surface unit is then decreased. However, the use of the electrografted polymers as described in this prior document is not evident, since the characteristics of the process do not make it possible to directly graft onto the surface a sufficient variety of useable functional groups. The term “direct grafting” is intended to mean the use of vinyl or cyclic monomers which comprise the functional groups that it is desired to attach to the surface, or which comprise original simple precursors (i.e. precursors which are not merely protected groups) of desired functional groups.

It has, moreover, already been proposed, in particular in patent application EP 0 665 275, to form a polymer film from electrograftable monomers according to a process during which the chain growth is interrupted with functional groups. Thus, according to this process, the functional groups of interest are not placed directly on the monomer which will be used for the electrografting, but essentially interrupt the growth with an inhibitor bearing the desired functional group. It is in particular noted that this process provides only one functional group per chain, and does not make it possible to have a large number of accessible groups, which is especially prejudicial when the probe molecule is large in volume. In addition, it has been demonstrated that the growth of the polymer chains of the surface is necessarily anionic (C. Bureau et al., Macromolecules, 1997, 30, 333), and it is probable that the free-radical inhibitors introduced according to this process are in the film at the end of synthesis because they are adsorbed and/or reduced on the surface of the electrode (they are in general electroactive), and not because they interrupt the chain growth.

Given the current understanding of the mechanisms of electrografting, and the prejudices associated therewith, it is possible to understand why those skilled in the art have not turned to the use of monomers having varied functional groups.

It appears to be accepted today that polymer films grafted by electrografting of activated vinyl monomers onto conducting surfaces are obtained by means of an electroinitiation of the polymerization reaction from the surface, followed by chain growth, monomer by monomer (C. Bureau, et al., 1997, mentioned above; C. Bureau and J. Delhalle, Journal of Surface Analysis, 1999, 6(2), 159 and C. Bureau, et al., Journal of Adhesion, 1996, 58, 101).

This polymerization reaction is represented in scheme A below:

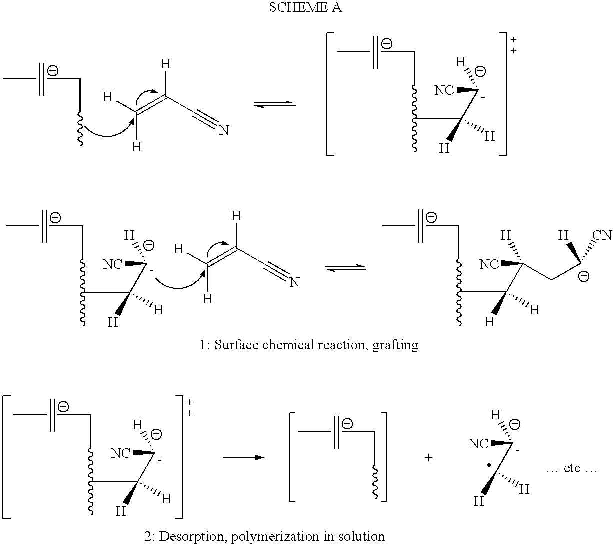

In this scheme, the grafting reaction corresponds to step 1, in which the growth occurs from the surface. Step 2 is the main parasitic reaction, which results in a nongrafted polymer being obtained.

The grafted chain growth therefore takes place by purely chemical polymerization, i.e. independently of the polarization of the conducting surface which gave rise to the grafting. This step is therefore sensitive to (it is in particular interrupted by) the presence of chemical inhibitors of this growth.

In the example of the reaction represented in scheme A above, in which the electrografting of acrylonitrile under cathodic polarization has been considered, the grafted chain growth takes place by anionic polymerization. This growth is interrupted in particular by protons, and it has been demonstrated that the proton content even constitutes the major parameter which controls the formation of polymer in solution, and also the information recovered during synthesis, and in particular the appearance of the voltammograms which accompany the synthesis (C. Bureau, Journal of Electroanalytical Chemistry, 1999, 479, 43). Traces of water, and more generally labile protons of protic solvents, constitute sources of protons that are prejudicial to the growth of the grafted chains. Even before the reaction mechanisms of electrografting of vinyl monomers were understood, this technical blocking point had been clearly identified by those skilled in the art.

For this reason, it appears to be impossible to envisage an electropolymerization reaction using monomers comprising functional groups that are proton sources (protic monomers).

Due to these severe limitations relating both to the solvents and to the types of monomers for synthesis, the electrografting of vinyl monomers onto electrically conducting or semiconducting surfaces remains a process allowing only the grafting of polymers that are relatively uninteresting from the point of view of the chemical functionalization of surfaces.

For this reason, electrografted polymer films have especially been used to produce passive functions: anti-corrosion or lubrication as has, for example, already been described in patent applications EP-A-0 038 244 and FR-A-2 672 661.

There exists therefore a need to be able to functionalize electrically conducting or semiconducting surfaces with organic layers having a large variety of functional groups, and also a large number of functional groups of interest accessible per surface unit, so as to ensure post-functionalization reactions that are faster than those currently available.

The applicant in particular gave itself the aim of solving the inorganic/organic interface problem so as to provide an electrically conducting or semiconducting support comprising a functionalized attachment zone or “molecular Velcro®” useful for attaching molecules of interest (probe molecules) or objects bearing a complementary function.

The technical details of the present invention, and also the examples of implementation, demonstrate that it is in particular possible, contrary to the teaching of the prior art, to obtain protic functional groups—either directly or indirectly—by electrografting, by making use of vinyl or cyclic monomers themselves bearing protic groups or precursors of protic groups, and more generally groups capable of reacting chemically with other organic functions.

A first subject of the present invention is therefore a solid support comprising at least one electrically conducting and/or semiconducting region containing a reducible oxide on its surface, characterized in that at least one zone of said surface is functionalized with an electrografted organic film obtained from electroactive organic precursors each comprising at least one functional group of interest, optionally in a mixture with electroactive organic precursors not comprising a functional group of interest, and in that the number of functional groups of interest accessible for the formation of a covalent, ionic or hydrogen bond with a complementary group within said film represents at least 90% of the total number of functional organic groups of interest.

One of the important specificities of the present invention is that a layer of functional groups of interest of which a large part is accessible for post-functionalization reactions—typically more than 90%—is produced by electrografting of organic coatings.

The electrografting of organic coatings makes it possible to produce interface bonds of covalent nature between an electrically conducting or semiconducting material and an organic material.

The functionalized organic film of the support in accordance with the present invention constitutes a veritable “molecular Velcro®” on which it is subsequently possible to call directly upon all the properties of the precursor which was electrografted, whether they are chemical or physical properties, in order to attach thereto various objects, such as for example (chemical or biochemical) molecules, polymers or cells, or even to obtain a function of bonding with respect to a macroscopic object, for example by chemical adhesion on the grafted precursor.

According to the present invention, the expression “functional group of interest that is accessible” is intended to mean a functional group that is sufficiently available, in particular in stearic terms, to form covalent bonds, ionic bonds or hydrogen bonds with a complementary group of size comparable to its own size. The molecule bearing this complementary functional group will be called probe molecule.

The term “complementary groups” is intended to mean functional groups of organic or organometallic chemistry which can react or interact with one another to give adducts that are sufficiently stable to be the source of an attachment between the two chemical entities—the coating and the probe molecule—which bear them. In this context, they may therefore be electrophilic groups or Lewis acids, such as carbonyls, carboxyls, isocyanates, epoxides, dienophiles, etc, capable of reacting with nucleophilic groups or Lewis bases such as amines, alcohols, thiols, dienes and polyenes, etc; H-bond donor groups such as amines, alcohols, thiols, carboxylic acids, etc, capable of interacting with lone-pair donors such as amines, alcohols, thiols, carboxyls, carbonyls, unsaturated bonds rich in electrons, etc; cationic groups, such as ammoniums, antimoniums, sulfoniums, diazoniums, etc, capable of interacting with anionic groups such as carboxylates, phosphates, phosphonates, sulfates, sulfonates, etc. A more exhaustive list of the pairs of complementary functional groups may be readily found in any organic chemistry monograph.

The accessibility of the functional groups of interest can be evaluated, quantitatively, by measuring for example the rate of conversion of these functional groups (for example by infrared, UV-visible, photoelectron spectroscopy, etc) when the coating containing these groups is reacted with a probe molecule containing a complementary functional group. If the probe molecule is small, it will in fact probably be able to react with all the functional groups of interest of the coating.

According to the invention, the expression “electroactive organic precursors not comprising a functional group of interest” is intended to mean any organic group optionally functionalized but incapable of forming covalent bonds, ionic bonds or hydrogen bonds with the given complementary group as defined above.

The advantage of a support comprising a coating having a large number of accessible functions is seen to an even greater extent when it is a question of attaching an object that is large to very large in size compared with the size of the functional group (typically, objects greater than a nanometer in size and, a fortiori, greater than about ten or about a hundred nanometers, or even a micrometer). In this situation, not all the accessible groups of interest of the coating will be used, but they will be sufficient in number to adapt as well as possible to the stearic constraints, and more generally to the topology, of the object that it is desired to attach to this coating.

According to the invention, the organic precursors are preferably chosen from:
polymerizable and electrograftable monomers bearing at least one organic functional group of interest. The electrografted organic film obtained is then a polymer;
polymerizable and electrograftable monomers bearing at least one functional group making it possible to simply obtain, by derivatization, the desired reactive functional organic group of interest. These are also referred to as monomers bearing synthons of the desired reactive functional groups of interest. The electrografted organic film obtained is then a polymer;
molecules, macromolecules and objects functionalized with monomers such as those described above. The organic film obtained is not then necessarily polymeric in nature.

Among the polymerizable monomers, mention may in particular be made of activated vinyl monomers and molecules that are cleavable by nucleophilic attack, corresponding respectively to formulae (I) and (II) below: 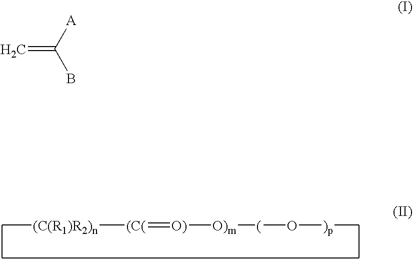
in which:
A, B, R₁ and R₂, which may be identical or different, represent a hydrogen atom, a C₁-C₄ alkyl radical, a nitrile radical or an organic function chosen from the following functions: hydroxyl, amine: —NHₓ with x=1 or 3, thiol, carboxylic acid, ester, amide: —C(═O)NH_{y} in which y=1 or 2, imide, imidoester, aromatic and in particular pyridine, styrene or halostyrene, acid halide: —C(═O)X in which X represents a halogen atom chosen from fluorine, chlorine or bromine, acid anhydride: —C(═O)OC(═O), nitrile, succinimide, phthalimide, isocyanate, epoxide, siloxane: —Si(OH)_{z} in which z is an integer between 1 and 3 inclusive, benzoquinone, carbonyldiimidazole, para-toluenesulfonyl, para-nitrophenyl chloroformate, ethylene and vinyl, or an organic group (or spacer arm) bearing at least one of the functions listed above, such as groups comprising several vinyl functions, for instance pentaerythritol tetramethacrylate; it being understood that at least one of A and B and that at least one of R₁ and R₂ represents one of said organic functions or an organic group bearing at least one of said functions;
n, m and p, which may be identical or different, are integers between 0 and 20 inclusive.

In the above notation, R₁ and R₂ are groups which depend on an index i not indicated, i being between 0 and n. This expresses the fact that the groups R₁ and R₂ may in fact be different from one (C(R₁)R₂) to another in the structure of the cyclic molecules of formula (II) above.

Among the activated vinyl monomers of formula (I) above, mention may in particular be made of methacryloyl succinimide, hydroxyethyl methacrylate (HEMA), methacrylonitrile, acrylonitrile, glycidyl acrylate and glycidyl methacrylate, acrylic acid, methacrylic acid, aminopropylmethacrylamide, aminohexylmethacrylamide, methacryloyl succinimide, acryloyl succinimide, methyl methacrylate, ethyl methacrylate, propyl methacrylate, butyl methacrylate, methyl cyanomethacrylate, methyl cyanoacrylate, 2- and 4-vinylpyridine and 4-chlorostyrene.

Among the molecules that are cleavable by nucleophilic attack, of formula (II) above, mention may in particular be made of ethylene oxide, substituted ethylene oxides, butyrolactone, caprolactones and in particular ε-caprolactone.

Among the molecules, macromolecules and objects functionalized with monomers, mention may be made of oligonucleotides, nucleic acid molecules such as DNA and RNA, oligopeptides, polypeptides such as poly-L-lysine, proteins such as avidin, streptavidin, antibodies, antigens, growth factors, fluorescent proteins such as for example the green fluorescent proteins (GFPs), ferredoxins, etc, oligosaccharides, polymers such as for example polyallylamine, polysaccharides and derivatives such as cellulose and modified celluloses, heparin, dextrans and substituted dextrans such as dextrans bearing carboxymethyl (CM), N-benzylmethylenecarboxamide (B) and sulfonate (S) groups, also called CMDBSs, telechelic polymers (i.e. polymers of any structure substituted at their ends with appropriate complementary functional groups, such as for example polyethylene glycol dimethacrylate), etc, fullerenes, functionalized carbon nanotubes, and cells; said molecules, macromolecules and said objects derivatized, totally or partially, with monomers corresponding to formula (I) or (II) described above.

According to the invention, the electrically conducting or semiconducting surface is preferably a stainless steel, steel, iron, copper, nickel, cobalt, niobium, aluminum (in particular when it is freshly brushed), silver, titanium, silicon (doped or undoped), titanium nitride, tungsten nitride or tantalum nitride surface, or a noble metal surface chosen from gold, platinum, iridium or platinum-iridium alloy surfaces; gold surfaces being particularly preferred according to the invention.

On the support in accordance with the invention, the density of the accessible functional groups of interest is preferably between 10⁴/μm² and 10¹⁰/μm².

A subject of the present invention is also a process for preparing a support as described above, characterized in that it consists in carrying out, in a single step, the electrografting of electroactive organic precursors onto at least one zone of at least one electrically conducting and/or semiconducting region containing a reducible oxide on its surface, of a solid support, by electrolysis, in an organic medium, of a composition containing, in said organic medium, at least one electroactive organic precursor comprising at least one functional group of interest, by bringing said composition into contact with said zone, the latter being subjected to a potential protocol during which it is brought, for all or part of the potential protocol (voltametric, potentiostatic, pulsed, etc), to a potential greater than or equal to a threshold electrical potential determined relative to a reference electrode, said threshold electrical potential being the potential beyond which the grafting of said precursors occurs, and in that a degree of accessibility of functional groups of interest of at least 90% (by number) is obtained:
a) by adjusting the potential protocol, and in particular the number of scans and the rate of scanning in a repeat protocol (voltametric pulsed, etc, scans) so as to produce a degree of grafting of less than or equal to 60%, and/or
b) by using a composition in which the functionalized electroactive organic precursors are present in a mixture with electroactive organic precursors not comprising a functional group of interest, the latter then representing from 0.1 to 95% of the total number of precursors present in said composition, and/or
c) by using electroactive organic precursors chosen from those in which the functional group of interest is borne at the end of a spacer arm.

By means of this process, it is possible to functionalize the surface with various organic groups and to produce a veritable “molecular Velcro®” on which it is subsequently possible to directly call upon all the properties of the polymer which was grafted, whether they are chemical or physical properties, so as to attach thereto various “objects”, such as for example (chemical or biochemical) molecules, polymers or cells, or even to obtain a bonding function with respect to a macroscopic object, for example by chemical adhesion on the grafted polymer.

This result is unexpected, given that any reactive group present on a vinyl monomer (other than the vinyl bond itself) is capable of carrying out parasitic reactions or even interrupting or preventing the chain growth during the electropolymerization of the grafted chains (see, for example: G. Deniau, et al., J. of Electroanalytical Chem., 1998, 451, 145).

However, the idea of the present invention holds in that it is not necessary to ensure long-chain growth on the surface in order to be able to benefit from the attachment of the functional groups of interest initially borne by the functionalized vinyl monomers. In this perspective, the parasitic reactions, or even the terminating reactions, which may appear due to the presence, on the initial vinyl monomer, of protic functional groups or functional groups that are reactive with respect to the growing end, and which are not protected, are relatively unimportant, provided that they do not consume all the functional groups of interest present on the precursors.

In particular, the electrografting of vinyl or cyclic monomers bearing varied organic groups of interest therefore makes it possible to envision the electrografted organic films as a means of obtaining, in one step, on the conducting and semiconducting surfaces, what could be attained with at least two steps when the procedure involved prior production of an oxide layer (for example by combining production of an oxide layer and chemical functionalization with a bifunctional adhesion primer). The process in accordance with the invention allows the formation of covalent bonds between the metal and the grafted polymer, which makes it possible to ensure the production of a layer that substantially contributes to the solidity of the interface.

According to this process, and in the case of variant a), the adjusting of the potential protocol makes it possible, in particular in the case of the polymers, to adjust the degree of grafting, i.e. the number of polymer chains grafted per surface unit: a moderate degree of grafting will allow, for example, the chains to be sufficiently spaced out to allow the thickness of the coating to be wetted with an appropriate solvent, and will also allow probe molecules to enter into the film of the coating. According to a preferred embodiment of this variant a), the degree of grafting is adjusted to a value of between 10 and 40%.

In the case of variant b), the functional groups of interest are spaced out from one another by carrying out the electrografting using a mixture of different monomers, only some of which bear the functional groups of interest that it is desired to have present on the final coating. The relative proportions of the various monomers then make it possible to adjust the number of functional groups of interest, and therefore their accessibility. According to a preferred embodiment of this variant b), the electroactive organic precursors not comprising a functional group of interest represent from 0.1 to 50% of the total number of precursors present in said composition.

The functionalized precursor (monomer or other) concentration conditions are variable from one precursor to another. It may, however, be considered that preferred concentrations are between 0.1 and 10 mol/l, and in particular between 0.1 and 5 mol/l, as regards the electroactive organic precursors comprising a functional group of interest. When electroactive organic precursors not comprising a functional group of interest are present in the organic composition (variant b)), these precursors are then present at a concentration preferably of between 10⁻³ and 18 mol/l, and even more preferably of between 10⁻³ and 9 mol/l.

According to variant c), it is also possible to improve the accessibility of the functional groups of interest by placing them at the end of a spacer arm, which may be, for example, a chain of a few carbon atoms. This spacer arm will have possibly been present directly on the precursors of the electrografted coating, or else added a posteriori. These spacer arms are in particular useful when the object to be attached to the coating is large in size: the attachment of a spacer arm to an electrografted coating is easier than that of a large object, since the (probe) molecule which contains the spacer arm is in general smaller than the object. It can therefore be attached to virtually all the accessible functional groups of interest of the electrografted coating, and replace them with groups that are even more accessible.

According to this process, the electrolysis is preferably carried out by polarization under voltametric conditions.

The organic medium used during this process is preferably chosen from dimethylformamide, ethyl acetate, acetonitrile and tetrahydrofuran.

This organic medium may also contain at least one support electrolyte which may in particular be chosen from quaternary ammonium salts such as perchlorates, tosylates, tetrafluoroborates, hexafluorophosphates, quaternary ammonium halides, sodium nitrate and sodium chloride.

Among these quaternary ammonium salts, mention may in particular be made, by way of example, of tetraethyl-ammonium perchlorate (TEAP), tetrabutylammonium perchlorate (TBAP), tetrapropylammonium perchlorate (TPAP) and benzyltrimethylammonium perchlorate (BTMAP).

A film of poly(methacryloyl succinimide) on gold is, for example, obtained by performing 10 voltametric scans of −0.4 to −2.8 V/(Ag+/Ag) at 50 mV/s on a gold surface immersed in a 0.5 mol/l solution of methacryloyl succinimide in DMF, in the presence of 5×10⁻² mol/l of TEAP. The succinimide functions are detected by infrared reflection-absorption spectroscopy (IRRAS) on the film obtained, after rinsing for 5 minutes with ultrasound. As is subsequently detailed in the examples of implementation, this grafted film readily allows the attachment of polyallylamine by reaction of the amine groups of the polyallylamine with the succinimide groups of the electrografted poly(methacryloyl succinimide).

It is observed, moreover, that a poly(methacryloyl succinimide) film can also be obtained at 0.18 mol/l in acetonitrile, both on gold and on platinum.

Alternatively, the formation of an electrografted film of poly(hydroxyethyl methacrylate) (PHEMA) on gold is observed by carrying out 10 voltametric scans of +1.0 to −3.0 V/(Ag+/Ag) at 50 mV/s on a gold surface immersed in a 0.4 mol/l solution of hydroxyethyl methacrylate in DMF, in the presence of 5×10⁻² mol/l of TEAP (tetraethylammonium perchlorate). It may be noted that this film is obtained with a monomer bearing nonprotected hydroxyl groups, whereas the prior art mentioned that it was necessary to protect these hydroxyl groups in order to carry out the HEMA electrografting (see in particular patent application EP-A-0 665 275). As is detailed in the examples of implementation, this electrografted PHEMA film readily reacts with diisocyanate groups, so as to obtain a post-functionalization of the surface, which shows that the chain growth, nevertheless hindered by the presence of the protic group, is not necessary for obtaining electrografted coatings, which can serve as a “molecular Velcro®”.

Finally, a subject of the invention is the use of the support in accordance with the invention as an adhesion primer (“molecular Velcro®”) for attaching molecules of interest (probe molecules) or objects bearing a complementary function.

According to a first advantageous embodiment of this use, the support in accordance with the invention can be used for attaching proteins (avidin, antibodies, growth factors, etc). The potential applications concern, for example, the production of bioactive surfaces (angioplasty, bioactive prostheses, etc) that promote cell adhesion and, optionally, recolonization; the production of surfaces which can be used for selective cell sorting (by attachment of antibodies specific for the wall of a given cell); the production of protein-chip matrices based on a support with conducting blocks.

According to a second advantageous embodiment of this use, the support in accordance with the invention can also be used for attaching nucleic acid molecules such as DNA, RNA or oligonucleotide molecules, for example for producing bioactive surfaces (antisense oligonucleotides) or attachment blocks for chemical or biochemical analysis chips, for instance nucleic acid chips such as DNA chips.

According to a third advantageous embodiment of this use, the support in accordance with the invention can also be used for attaching oligosaccharides, and more generally biomaterials (biocompatible polymers such as polysaccharides, for instance dextrans, ceramics, etc), for example for producing biocompatible surfaces or surfaces with encapsulating properties.

Finally, according to a fourth advantageous embodiment of this use, the support in accordance with the invention can also be used for bonding objects to conducting or semiconducting surfaces by means of surface chemical reactions.

Besides the above provisions, the invention also comprises other provisions which will emerge from the following description, which refers to examples of preparations of supports in accordance with the invention comprising a surface coated with a film of poly(methacryloyl succinimide), of poly(hydroxyethyl methacrylate) or of polymethacrylonitrile (PMAN), an example illustrating the use of a support coated with an electrografted poly(methacryloyl succinimide) film as an adhesion primer for attaching polyallylamine, to an example illustrating the use of a support covered with a poly(hydroxyethyl methacrylate) film as an adhesion primer for forming a carbamate, and to examples illustrating the use of a support comprising a polymethacrylonitrile film as an adhesion primer for attaching various molecules or macromolecules, and also to FIGS. 1 to 16 in the appendix, in which:

FIG. 1 represents the IRRAS spectrum of a gold surface coated with an electrografted poly(methacryloyl succinimide) film;

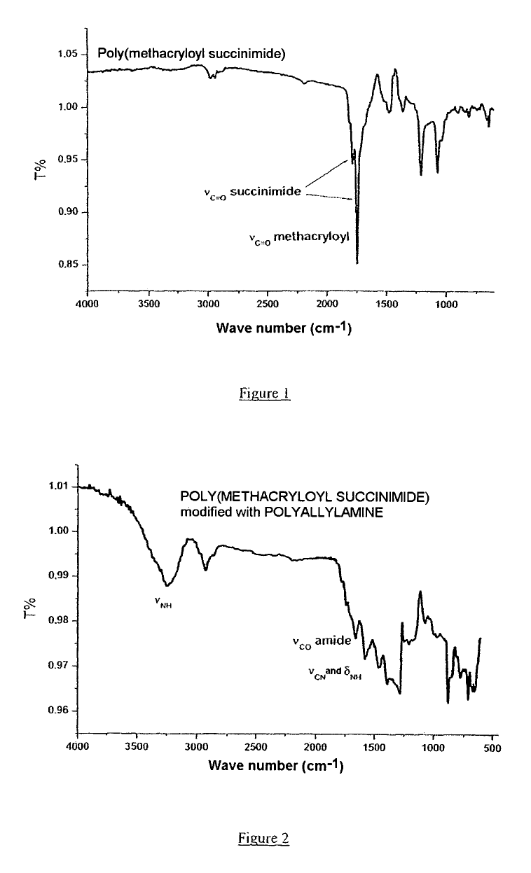

FIG. 2 represents the IRRAS spectrum of a gold surface coated with a poly(methacryloyl succinimide) film post-functionalized with polyallylamine;

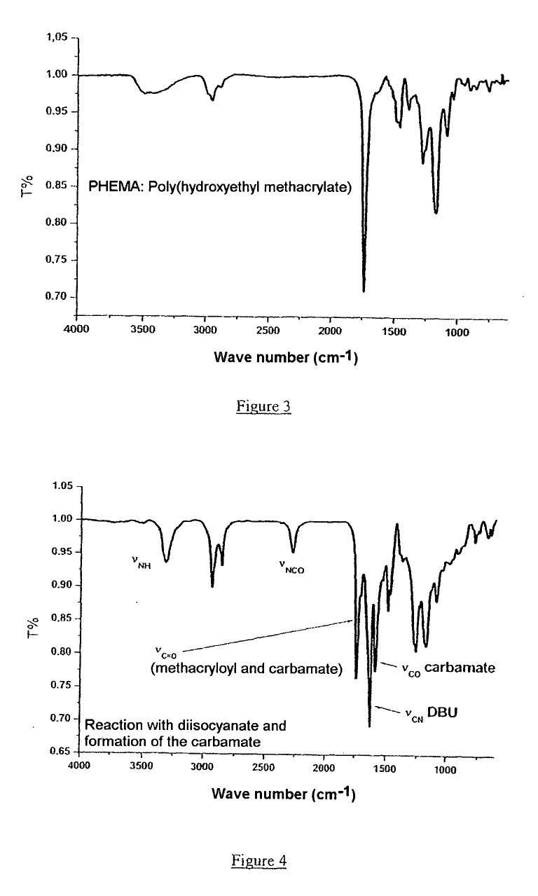

FIG. 3 represents the IRRAS spectrum of a gold surface coated with a poly(hydroxyethyl methacrylate) film;

FIG. 4 represents the IRRAS spectrum of a gold surface coated with a poly(hydroxyethyl methacrylate) film after reaction with diisocyanatohexane and formation of a carbamate;

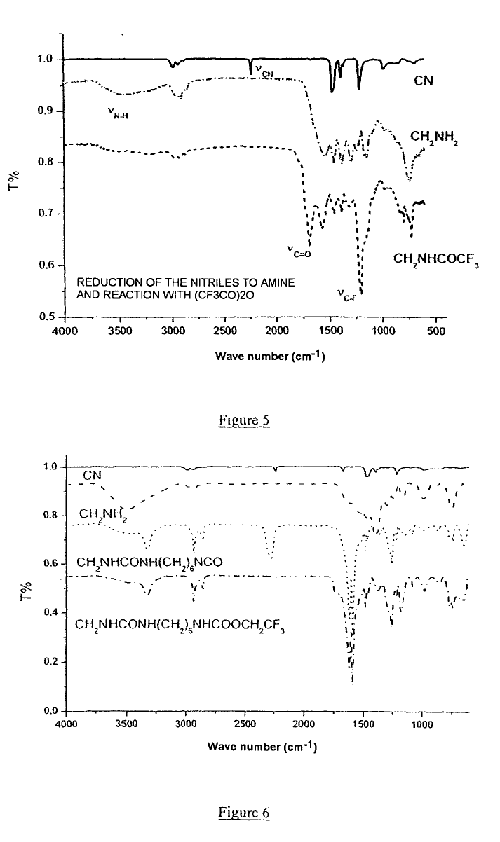

FIG. 5 represents the IRRAS spectra of a gold surface coated with an electrografted PMAN film (top spectrum), after reduction of the nitrile groups to amines (middle spectrum) and after reaction of these amine groups with trifluoroacetic anhydride to form an amide (bottom spectrum);

FIG. 6 represents the IRRAS spectra of a gold surface coated with an electrografted PMAN film (CN), after reduction of the nitrile groups to amines with lithium aluminum hydride (CH₂NH₂), after reaction of these amine groups with 1,6-diisocyanatohexane to form urea (CH₂NHCONH(CH₂)₆NCO), and after reaction with trifluoroethanol to form the carbamate (CH₂NHCONH(CH₂)₆NHCOOCH₂CF₃);

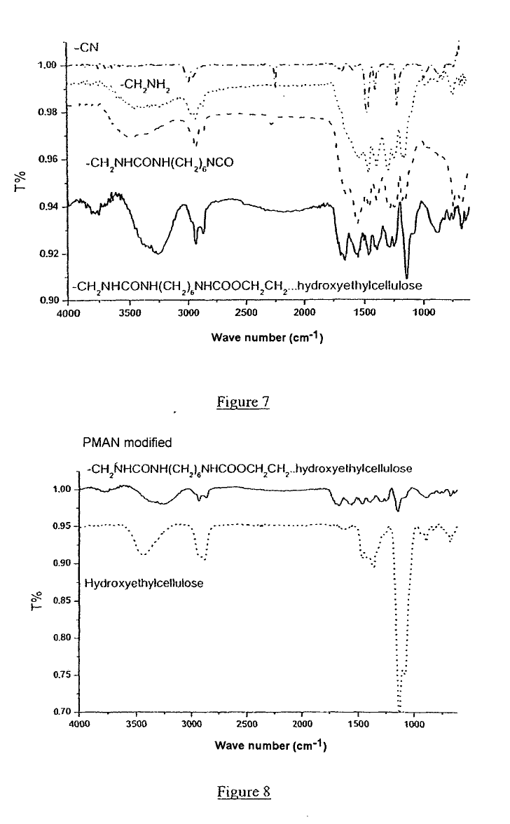

FIG. 7 represents the IRRAS spectra of a gold surface coated with an electrografted PMAN film (CN), after reduction of the nitrile groups to amines with lithium aluminum hydride (CH₂NH₂), after reaction of these amine groups with 1,6-diisocyanatohexane to form urea (CH₂NHCONH(CH₂)₆NCO) and after reaction with hydroxyethylcellulose to form the corresponding carbamate;

FIG. 8 represents the IRRAS spectra of a gold surface coated with an electrografted PMAN film onto which hydroxyethylcellulose has been grafted, and that of a KBr disk containing hydroxyethylcellulose;

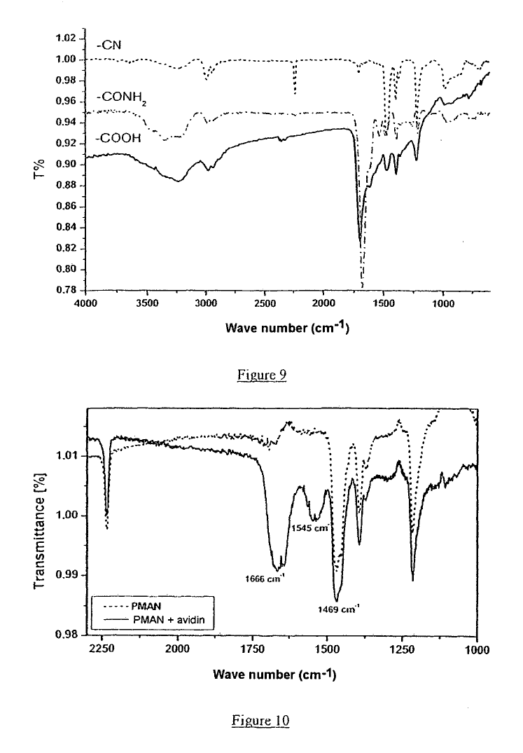

FIG. 9 represents the IRRAS spectra of a gold surface coated with an electrografted PMAN film after hydrolysis of the nitrile groups to amide (acid treatment), and then to carboxylic acid (basic treatment);

FIG. 10 represents the IRRAS spectra of a gold surface coated with an electrografted PMAN film onto which avidin has been grafted;

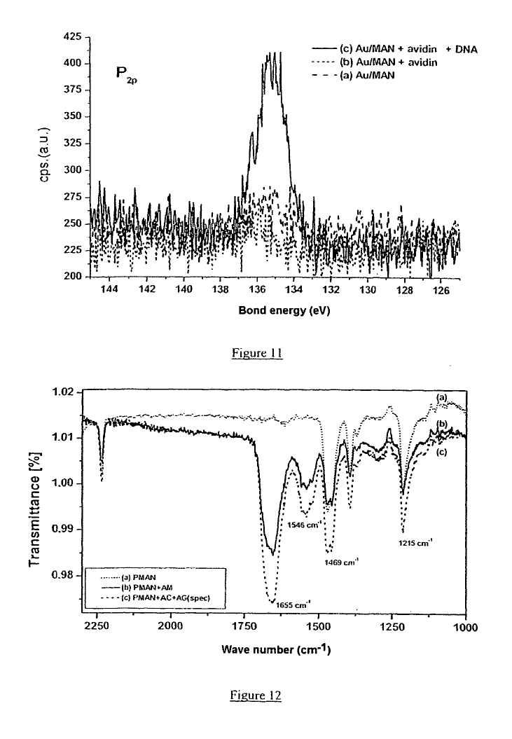

FIG. 11 represents the region P₂ₚ of the spectrum determined by X-ray photoelectron spectroscopy (XPS) of a gold surface coated with an electrografted PMAN film (a); after attachment of avidin (b) and after attachment of avidin and of an oligonucleotide biotinylated at its 5′ end;

FIG. 12 represents the IRRAS spectra of a gold surface coated with an electrografted PMAN film (a), to which an anti-rabbit IgG antibody has been attached (b), treated with a solution of specific antigen (c);

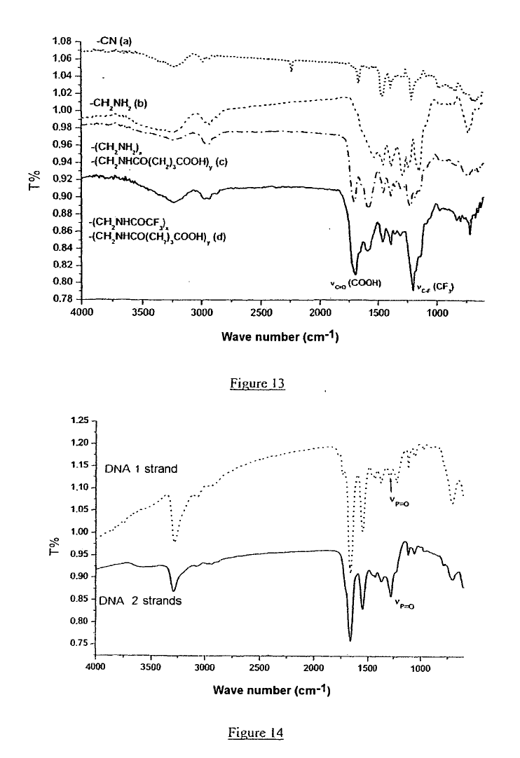

FIG. 13 represents the IRRAS spectra of a gold surface coated with an electrografted PMAN film (a), on which the nitrile groups have been reduced (b), treated with glutaric anhydride to form amides (c), and then with trifluoroacetic anhydride (d);

FIG. 14 represents the IRRAS spectra of FIG. 13 (d), after reaction with a single-stranded oligonucleotide aminated in the 5′ position, and then with a second oligonucleotide complementary to the first;

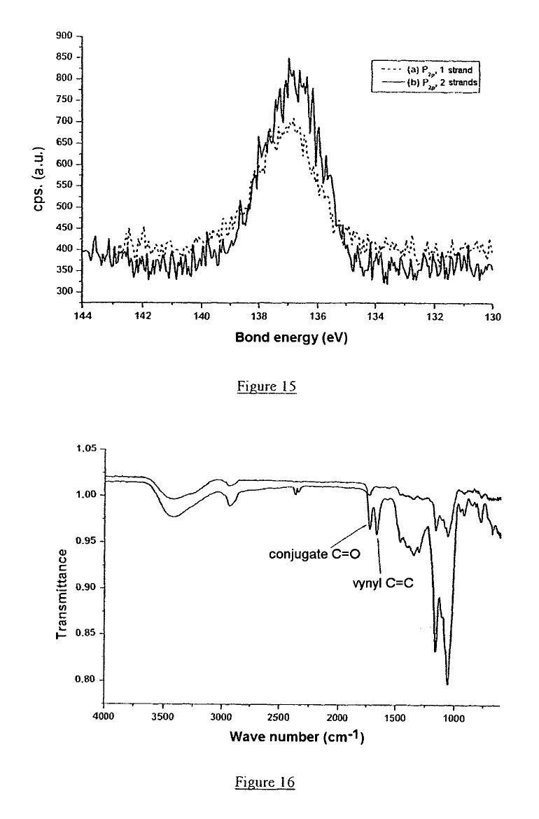

FIG. 15 represents the region P₂ₚ of the XPS spectrum of the film of FIG. 13 (d) after reaction with a single-stranded oligonucleotide aminated in the 5′ position, and then with a second oligonucleotide complementary to the first; and

FIG. 16 represents the IRRAS spectrum of a film of electrografted dextran functionalized with glycidyl methacrylate groups (top spectrum) and the spectrum of the dextran functionalized with glycidyl methacrylate groups before electrografting (bottom spectrum).

### EXAMPLE 1

### Attachment of Polyallylamine by Means of an Electrografted Poly(Methacryloyl Succinimide) Film

This example illustrates both the electrografting of a monomer bearing a functional group of interest which can be involved in the functionalization with an organic molecule (succinimide group, electrophile) and the post-functionalization reaction itself, via the reaction of amines (nucleophiles) with the succinimide groups of the electrografted polymer. The probe bearing the amine groups is a polymer, polyallylamine, and the post-functionalization reaction is therefore a polymer-on-polymer reaction, which illustrates the great accessibility of the succinimide groups of the electrografted coating.

a) Formation of an Electrografted Poly(Methacryloyl Succinimide) Film

10 voltametric scans of −0.3 to −2.5 V (Ag⁺/Ag) are carried out at 50 mV/s on a gold surface immersed in a 0.18, 0.25 or 0.5 mol/l solution of methacryloyl succinimide (MASU) in DMF, in the presence of 5×10⁻² mol/l of TEAP. A poly(methacryloyl succinimide) film is obtained, as proved by the IRRAS spectrum of the surface represented in FIG. 1 in the appendix, which exhibits the characteristic carbonyl bands at 1782 and 1746 cm⁻¹ (transmittance as % as a function of the wavelength in cm⁻¹).

This IRRAS spectrum was determined after rinsing with acetone for 5 minutes with ultrasound.

Table I below summarizes the IRRAS characteristics (intensity of the band C═O of the succinimide groups) as a function of the synthesis conditions.

In this table, VC indicates a scan under voltametric conditions; the potential limits indicated are located relative to a silver electrode.

TABLE I

| TEAP/DMF | | Conditions for | |
|---|---|---|---|
| electrochemical | | standard synthesis | IRRAS charac. |
| medium | | of the film | % C═O |
| 0.18 | M MASU | 5*1 VC, 50 mV/s | 7.78 |
| | | from −0.6 to −2.8 | |
| 0.18 | M MASU | 5 VC, 50 mV/s | 7.13 |
| | | from −0.6 to −2.8 | |
| 0.25 | M MASU | 10 VC, 50 mV/s | 17.2 |
| | | from −0.3 to −2.5 | |
| 0.5 | M MASU | 10 VC, 50 mV/s | 45 |
| | | from −0.6 to −2.5 | |
| 0.5 | M MASU | 10 VC, 50 mV/s | 55 |
| | | from −0.4 to −2.8 | |

b) Post-Functionalization Reaction: Attachment of the Polyallylamine

20 ml of deionized water, and then 0.5 ml of a 20% by weight solution of polyallylamine in deionized water, are introduced into a ground tube equipped with a magnetic stirrer. The gold slide bearing an electrografted poly(methacryloyl succinimide) film, obtained according to the protocol above, is then introduced. The slide is left, with stirring, for 1 hour 30 min. at ambient temperature.

It is then removed from the tube, rinsed with jets of deionized water, and then with ultrasound in deionized water for 2 minutes, and finally dried by nitrogen blowing.

By IRRAS (FIG. 2) a decrease in the characteristic bands of the succinimide groups at 1746 and 1782 cm⁻¹ is observed, along with the appearance of the characteristic bands of polyallylamine, and in particular the amide band ν_{CO} at 1656 cm⁻¹, the elongation ν_{CN} and deformation δ_{NH} bands at 1574 cm⁻¹, and the elongation band ν_{NH} at 3254 cm⁻¹.

### EXAMPLE 2

### Formation of a Carbamate by Reaction of Diisocyanatohexane with the Hydroxyl Groups of an Electrografted Poly(Hydroxyethyl Methacrylate) (PHEMA) Film

This example illustrates the electrografting of a monomer bearing hydroxyl groups (HEMA), and the formation of a PHEMA film, and also the use of the hydroxyl groups of the PHEMA for reacting with the isocyanate groups of diisocyanatohexane so as to form a carbamate. It also illustrates the great accessibility of the hydroxyl groups of the electrografted polymer with respect to the probe molecule which is constituted by the diisocyanatohexane, since all the groups are converted in the reaction.

a) Formation of a PHEMA Film

A PHEMA film is produced on gold by means of 10 voltametric scans at 50 mV/s from −2.4 to +1 V (Ag⁺/Ag) on a gold surface immersed in a 2.7 mol/l solution of hydroxyethyl methacrylate (HEMA) in DMF, in the presence of 5×10⁻² mol/l of TEAP. The IRRAS spectrum of the film obtained is given in FIG. 3. The presence of the characteristic carbonyl band at 1737 cm⁻¹ is noted. A band is also observed at around 3500 cm⁻¹, due to the hydroxyl groups of the hydroxyethyl arms of the polymer.

b) Post-Functionalization Reaction: Attachment of the Diisocyanatohexane

30 ml of toluene dried on 4 Å molecular sieves, 1.5 ml of 5% by volume diisocyanatohexane in toluene and 2 drops of 1,8-diazabicyclo[5,4,0]undec-7-ene (DBU) are introduced into a dry ground tube.

The gold slide coated with an electrografted PHEMA film obtained above in the preceding step, pre-soaked in and coated with a layer of dry toluene, is then introduced.

The tube is closed, and then left to react at ambient temperature under argon for 142 hours. The slide is removed, rinsed with dry toluene and then with dry acetone by means of jets. It is then dried with nitrogen.

The IRRAS spectrum of the slide determined after reaction with diisocyanatohexane and formation of the carbamate is represented in FIG. 4.

The appearance of the ν_{NH} elongation bands at 3330 cm⁻¹ and of the N═C═O isocyanate band at 2264 cm⁻¹ is observed. The band at 1623 cm⁻¹ is probably due to the presence of residual DBU. The disappearance of the band at around 3500 cm⁻¹ due to the hydroxyl groups is also observed, which shows that the conversion of these groups was quantitative, and that they were therefore all accessible for the probe molecule.

### EXAMPLE 3

### Obtaining Amine Groups on a Gold Slide by Reduction of the Nitriles of an Electrografted Polymethacrylonitrile Film

This example illustrates the use of the nitrile groups of a polymethacrylonitrile (PMAN) film as precursors of amine groups, and the reactivity of these amine groups by formation of amides with trifluoroacetic anhydride. Here again, the functionalization reaction is quantitative, which shows that the nitrile, and then amine, groups are very accessible.

a) Preparation of a Gold Slide Coated with an Electrografted PMAN Film

A PMAN film is produced on gold by carrying out 10 voltametric scans from −0.5 to −2.7 V/(Ag⁺/Ag) at 50 mV/s on a gold surface immersed in a 2.5 mol/l solution of methacrylonitrile in DMF, in the presence of 5×10⁻² mol/l of TEAP. The nitrile groups of the polymer formed are identified by means of the band at 2235 cm⁻¹ in IRRAS.

b) Post-Functionalization Reaction: Formation of Amides with Trifluoroacetic Anhydride

The slide coated with the PMAN film obtained above in step a), blown with nitrogen, is introduced into a tube equipped with a septum. The septum is closed, and then 20 ml of pyridine dried on a molecular sieve, and 1 ml of a solution of lithium aluminum hydride, LiAlH₄, at 1 mol/l in tetrahydrofuran (THF) dried on a molecular sieve, are introduced under argon using a purged syringe. The slide is left in the reaction medium for 2 minutes at 70° C. The slide is then rinsed with pyridine by soaking for 5 minutes, and then with jets of deionized water, dried by nitrogen blowing, treated with ultrasound for 1 minute in a 1 mol/l sodium hydroxide solution, rinsed with deionized water, and then dried by nitrogen blowing.

FIG. 5 in the appendix represents the IRRAS spectra of the gold slide coated with an electrografted PMAN film (top), after reduction of the nitrile groups to amine with lithium aluminum hydride (middle), and after reaction of these amine groups with trifluoroacetic anhydride so as to form the amide (bottom).

The disappearance of the nitrile elongation band at 2235 cm⁻¹ is observed, along with the appearance of the NH₂ group ν_{NH} elongation band between 3250 and 3450 cm⁻¹, the CH₂(NH₂) asymmetric elongation band at 2929 cm⁻¹, and the NH₂ deformation band at 1642 cm⁻¹, as a characteristic of the formation of polyallylamine.

### EXAMPLE NO. 4

### Reactivity of the Amine Groups Formed According to the Embodiment of Example No. 3

The aim of this example is to verify that the amine groups which were produced above in Example 3 are accessible and conserve their reactivity. This is realized by amidation of the amine functions, according to the procedure described in J. Org. Chem., 1989, 54, 2498, and readapted in the present case for a reaction on a gold surface.

20 ml of a 0.35 mol/l solution of trifluoroacetic anhydride in THF are introduced into a tube. The slide obtained at the end of Example 3 is dipped for 2 minutes at ambient temperature under argon (septum). The slide is removed, rinsed with dry THF and then dried by nitrogen blowing.

The coating obtained is analyzed by IRRAS (not represented), and is very characteristic of the formation of amide groups from amines: the occurrence of the amide band at 1694 cm⁻¹, the CN elongation and N—H deformation band at 1572 cm⁻¹, and the C—F elongation band at 1209 cm⁻¹ with, at around 1250 cm⁻¹, the CNH deformation band, is observed. At the same time, the virtually complete disappearance of the amine elongation band at around 2929 cm⁻¹ is observed.

### EXAMPLE 5

### Reactivity of the Amine Groups Formed in Example 3; Reaction with 1,6-Diisocyanatohexane, Formation of Urea

This example illustrates the reaction of the amine groups formed in Example No. 3 with a bifunctional coupling agent, so as to form a urea. The urea formed at the surface is used to attach an alcohol thereto. The procedure for synthesizing the urea at the surface is adapted from Org. Synth., 1988, VI, 951.

30 ml of a 5% by volume solution of 1,6-diisocyanatohexane (ONC—(CH₂)₆—NCO) in dry toluene (dried on 4 Å molecular sieves) are introduced into a tube. A gold slide bearing an electrografted film containing amine groups, and as obtained from Example No. 3, coated with a layer of dry toluene, is introduced. The slide is left to react for 22 hours at ambient temperature with magnetic stirring, under argon. It is removed from the tube, rinsed with jets of dry toluene, and then dried by nitrogen blowing.

The film obtained is in fact reacted with trifluoroethanol according to the following protocol: 30 ml of dry toluene (4 Å molecular sieve), 1.5 ml of trifluoroethanol, and 3 drops of DBU are introduced into a tube. The slide bearing the electrografted film modified with 1,6-diisocyanatohexane, coated with a layer of dry toluene, is placed therein. The slide is left in contact with the solution, under argon and with magnetic stirring for 88 hours at ambient temperature. The slide is removed, rinsed with dry toluene and then with acetone, with deionized water and, finally, with acetone by means of jets, and dried by nitrogen blowing.

FIG. 6 in the appendix shows the IRRAS spectra of the gold slide coated with an electrografted PMAN film (CN), after reduction of the nitrile groups to amine with lithium aluminum hydride (CH₂NH₂), after reaction of these amine groups with 1,6-diisocyanatohexane so as to form urea (CH₂NHCONH(CH₂)₆NCO), and after reaction with trifluoroethanol so as to form the carbamate (CH₂NHCONH(CH₂)₆NHCOOCH₂CF₃).

The ν_{N—H} elongation bands at 3330 cm⁻¹, the O═C═N elongation band at 2271 cm⁻¹ and also the urea bands at 1633 and 1576 cm⁻¹ are observed, proof of the reaction of the initial amine groups with at least one of the two isocyanate groups of the 1,6-diisocyanatohexane. The O═C═N band shows, in addition, that some of the isocyanate sites remain available, which is proved through the use of these groups to react with an alcohol.

After reaction with the trifluoroethanol so as to form the carbamate (CH₂NHCONH(CH₂)₆NHCOOCH₂CF₃), the IRRAS spectrum of the slide obtained also shows the carbamate C═O band at 1722 and at 1590 cm⁻¹ (mixed up with that of the urea), the CH₂O band (CF₃CH₂O—) at 1256 cm⁻¹, and the C—F bond elongation bands at 1179 cm⁻¹. The disappearance of the NCO band at 2271 cm⁻¹ is also noted.

Here again, the conversion of the functional groups successively realized on the coating is quantitative, which shows their great accessibility.

### EXAMPLE 6

### Use of the Functional Groups of an Electrografted Polymer Film for Attaching Hydroxyethylcellulose

This example illustrates the fact that the urea formed in Example 5 above also allows the attachment of hydroxyethylcellulose, and more generally of polysaccharides. This route illustrates the reaction of a macromolecule having a complex three-dimensional structure, the attachment of which is made possible by the great accessibility of the functional groups of interest on the electrografted coating. It is advantageous since it allows the attachment of polymers or of macromolecules which are difficult to attach to electrically conducting surfaces, and in particular to metals, and the value of which is to open up the pathway to the production of biomimetic surfaces (heparin, modified dextrans, hyaluronic acid, etc.) on metals, and of a module for attachment of complex biological molecules of interest (DNA, proteins, growth factors, etc.).

A gold slide coated with an electrografted film modified with 1,6-diisocyanatohexane and bearing free isocyanate groups is produced, as described in Example 5 above.

30 ml of DMF dried on a 4 Å molecular sieve are introduced into a tube. The solution is degassed by argon sparging for 10 minutes. 0.6 g of hydroxyethylcellulose is then introduced, and the solution is heated at 60° C., in order to obtain dissolution, with magnetic stirring for 15 minutes. 5 drops of DBU are then added and the slide bearing isocyanate groups, coated with its synthesis solution (toluene and 1,6-diisocyanatohexane in excess), is then introduced. The slide is left to react for 46 hours at 50° C. under argon and with magnetic stirring. The slide is removed and rinsed for one hour in deionized water with magnetic stirring.

The IRRAS spectra of the support thus obtained is represented in FIG. 7 in the appendix. In this figure, the spectrum of a gold slide coated with an electrografted PMAN film (CN), that of the slide after reduction of the nitrile groups to amine with lithium aluminum hydride (CH₂NH₂), and then after reaction of these amine groups with 1,6-diisocyanatohexane so as to form urea (CH₂NHCONH(CH₂)₆NCO) and, finally, after reaction with hydroxyethylcellulose so as to form a carbamate (CH₂NHCONH(CH₂)₆NHCOOCH₂CH₂ hydroxyethyl-cellulose) can be seen. The secondary carbamate band is observed at 1715 cm⁻¹, along with the characteristic bands of hydroxyethylcellulose between 1200 and 1000 cm⁻¹, which correspond to the ether (COC) and alcohol (OH) group elongation bands.

FIG. 8 shows, for comparison, the spectrum of the film obtained with the gold slide in accordance with the invention and that of a KBr disk containing hydroxyethylcellulose. This spectrum confirms the attachment of the hydroxyethylcellulose to the support of the invention.

### EXAMPLE 7

### Electrografted PMAN Film that is a Precursor of Amide and Carboxylic Acid Groups on Conducting and Semiconducting Surfaces

This example illustrates the fact that a PMAN film such as that obtained above in Example 3 can be used as a simple precursor of amide and carboxylic acid groups on metal surfaces. This conversion has the advantage of readily resulting in the formation of reactive groups that are different from the starting film, but also of allowing the simple production of hydrophilic surfaces from hydrophobic electrografted films (which facilitates in particular the use of the films as hydrophilic compound adhesion primers, and can be useful in the production of coatings that are more readily accepted in biomedical applications).

The nitrile functions were modified to carboxylic acid functions in two steps:
an acid step:
   R—CN+H₂SO₄→R—CONH₂
a basic step:
   R—CONH₂+NaOH→R—COOH

According to the reaction time, it is possible to have a conversion of a few % to 100%. These treatments are accompanied by a considerable loss of thickness. After the acid treatment, the treated area is hydrophilic. Water thoroughly wets the modified part and forms “a layer” on the surface.

The two reactions are carried out under atmospheric pressure at 100° C. (internal temperature) in open beakers or flasks. After each treatment, the slides are rinsed by dipping for 5 minutes in water, and are then dried by nitrogen blowing.

The solutions used are as follows:
acid solution:
21 ml of H₂SO₄ at a minimum of 95%
3.5 g of NaHSO₄: solution at approximately 37 N;
basic solution:
18 g of NaOH/25 ml H₂O: solution at 18 N.

A partial treatment is obtained by dipping the slide in the acid medium for a time equal to or less than 5 seconds, and by dipping it in the basic medium for 5 to 10 seconds. A treatment of 30 seconds in the 2 media results in complete disappearance of the nitrile functions, which corresponds to their complete conversion.

An IRRAS analysis is performed before and after each step: gold slide coated with the electrografted PMAN film before and after hydrolysis of the nitrile groups to amide (acid treatment) and then after conversion to carboxylic acid functions (basic treatment).

The IRRAS spectra obtained are given in FIG. 9 in the appendix.

The analysis of these spectra reveals, for the acid treatment, the formation of amide bands at 1680 cm⁻¹ (C═O elongation), and of NH₂ group deformation bands at 1605 cm⁻¹. After basic treatment, it is noted that the carbonyl band has shifted to 1700 cm⁻¹, which corresponds to carboxylic acid groups that are probably dimerized.

### EXAMPLE 8

### Attachment of Avidin to an Electrografted PMAN Film

This example illustrates that the nitrile groups of an electrografted PMAN film can be used for the covalent attachment of proteins. It is in fact known that nitrites can react with alcohols to give iminoethers (Pinner synthesis, cf.: P. L. Compagnon, M. Miocque, Annales de Chimie, 1970, 5, 23) according to the following reaction:
R—CN+R′—OH→R—C(OR′)═NH

The same type of reaction is also known for amines and thiols. As in Example 6 above, the attachment of a macromolecule having a complex three-dimensional structure is achieved, and is only possible due to the great accessibility of the nitrile functions of the electrografted polymer. In the following example, it is illustrated that this accessibility is such that it even allows the attachment of the protein in a conformation in which it conserves its activity, by reaction with a molecule bearing a biotin fragment having a very high affinity for avidin.

30 ml of a 2 mg/l solution of avidin in a phosphate buffered saline (PBS) of pH 7.2 are introduced into a tube equipped with a septum. A gold slide coated with an electrografted PMAN film as prepared above in Example 3 is placed therein. The slide is left to react for 15 hours at a temperature of 4° C. It is then removed and rinsed with deionized water.

The IRRAS spectrum of the slide thus obtained is given in FIG. 10 in the appendix.

Analysis of this spectrum shows the presence of the amide bands I (1666 cm⁻¹) and II (1545 cm⁻¹), and also the bands of the backbone (1469 cm⁻¹) that are characteristic of the protein.

### EXAMPLE 9

### Verification of the Activity of the Avidin Attached to an Electrografted PMAN Film

This example illustrates that the avidin attached according to the protocol of Example 8 is active, by using it as a point of attachment of a biotinylated oligonucleotide (ODN). The ODN used is the 15-mer below:
Biotin-5′-GCTTGCTGAAGTTCG-3′ (Biotin-SEQ ID No. 1)

The slide obtained according to the process of Example 8 is immersed in a 25 μM solution of this ODN in a PBS buffer (pH 7.2), in a tube. The slide is reacted at ambient temperature for 15 hours, removed, and rinsed several times with jets of deionized water.

The presence of the ODN is detected by X-ray photoelectron spectroscopy (XPS). The curves corresponding to this analysis and also to that of a slide coated with a simple electrografted PMAN film and to that of the slide obtained above in Example 8 (after attachment of avidin) are given in FIG. 11 in the appendix. The region P₂ₚ shows the presence of the phosphorus atoms of the phosphate groups of the ODN bases.

These results show that the slide prepared in accordance with Example 8 makes it possible to attach avidin in a conformation in which it conserves its activity, by reaction with a molecule bearing a biotin fragment having very high affinity for avidin.

### EXAMPLE 10

### Attachment of Antibodies to an Electrografted PMAN Film and Verification of its Activity

This example illustrates the fact that an electrografted film can be used as a primer for attaching molecules having a complex three-dimensional structure, and where the structure is determinant in the properties of the molecule. The great accessibility of the functional groups of interest present on the surface in fact enables minimum distortion of the probe protein, which can thus conserve an active conformation.

For this, an antibody, the anti-rabbit IgG immunoglobulin, is attached. The activity and the specificity of this antibody are then verified by reaction, firstly, with a specific antigen (rabbit IgG) and, secondly, with a nonspecific antigen (sheep IgG).

It should be noted that the attachment of an antibody opens up in particular the pathway to the attachment of a cell via electrografted polymers.

In order to allow the attachment of an antibody to a surface (for example to the transducer of a sensor), it is in general necessary, beforehand, to modify the electrode. As a result, many superficial groups can be created, but they must allow the coupling of immunoglobulins; thus, three types of functions drew our attention: amine, alcohol, cyano.

Amine and alcohol functions are often used to attach antibodies to a surface. Many commercial coupling agents thus exist for creating covalent bonds between superficial functions and those of immunoglobulins.

On the other hand, the cyano function allows direct attachment of the biomolecule. This method is original and has never been used to attach an immunoglobulin to a surface, and in particular to a conducting surface.

Antibodies contain various functions: amine (NH₂), acid (COOH), hydroxyl (OH) and disulfide bridges (S—S) which can bring about their attachment to surfaces. The amine and acid functions originate from the amino acids, that are constituents of the antibodies and are distributed throughout the protein. They are therefore several possible sites of attachment that allow easy but non-localized coupling, which may result in inactivation of the antibody (denaturation) with respect to the antigen. The amine and acid functions make it possible to graft the whole antibody to a surface. On the other hand, it is necessary to cleave the disulfide bridges (S—S) and therefore to generate thiol functions (SH). It is then the FAB′ fragments which are attached. The layer of biomolecules thus obtained is more dense in terms of reactive sites and, in addition, the antibodies are oriented since the thiol functions are present in the remaining constant portion. The latter characteristic is important since the antibody does not attach via one of its active sites. The immunoglobulins thus immobilized have less of a risk of being denatured and inactivated with respect to the antigens.

Except in the case of the cyano functions, it is essential to use a coupling agent which makes it possible to covalently link the functions of the surface and of the antibody. The fact that the electrografted films make it possible both to provide a primer layer and to offer functional groups of interest that are immediately available for the attachment of biological probe molecules is illustrated here.

A 2 mg/l solution of anti-rabbit IgG in PBS buffer (pH 7.2) is introduced into a tube. A gold slide coated with an electrografted PMAN film as prepared above in Example 3 is immersed in this solution. The slide is left to react for 15 hours at 4° C., and is then removed and rinsed with jets of deionized water and dried by nitrogen blowing.

The slide thus treated is again immersed in a solution of specific antigen (rabbit IgG) at 2 mg/l in PBS buffer, and left at ambient temperature for 15 hours. It is then removed, rinsed with jets of deionized water, and dried by nitrogen blowing.

The slide is analyzed by IRRAS before and after treatment with the antibody and also after treatment with the antigen.

The IRRAS spectra thus obtained are given in FIG. 12 in the appendix.

Analysis of these spectra reveals the amide bands I (1655 cm⁻¹) and II (1546 cm⁻¹), and also the bands of the protein backbone at 1469 cm⁻¹.

An increase in the amide bands I (1655 cm⁻¹) and II (1546 cm⁻¹), and in the bands of the protein backbone at 1469 cm⁻¹, is also observed, proving that the amount of proteins attached to the surface has increased (a virtual doubling of the intensity of these bands is noted under the effect of the coupling with the antigen, the size of which is approximately the same as that of the antibody).

This result is all the more probative since, when a slide coated with antibody (anti-rabbit IgG) is treated, under the same conditions, in a solution containing a nonspecific antigen (sheep IgG), only a very slight increase in the above characteristic bands (probably due to nonspecific adsorptions) is observed on the IRRAS spectrum (not represented).

### EXAMPLE 11

### Attachment of DNA to an Electrografted PMAN Film

This example illustrates the attachment of oligonucleotides (ODNS) to the reactive functions of an electrografted polymer, according to an alternative pathway to that seen in Example 9 above.

For this, the carboxylic acid functions of an electrografted polymer are used so as to react them with the amine functions of a single-stranded ODN bearing an amine function at its 5′ end:
H₂N-5′-GCTTGCTGAAGTTCG-3′-(H₂N-SEQ ID No. 1)

The attachment of this strand is then revealed by hybridization with the nonfunctionalized complementary strand:
5′-CGAACGACTTCAAGC-3′ (SEQ ID No. 2)

In order to illustrate here the possible use of spacer arms, a prior complementary functionalization of the film is carried out: the starting material is an electrografted PMAN film, on which the nitrites are reduced to amines, for example as indicated in Example 3. The amines are reacted with glutaric anhydride so as to obtain carboxylic acid functions, according to the following protocol: 30 ml of THF dried on a molecular sieve (4 Å) are introduced into a tube, and 1 g of glutaric anhydride is added thereto. The slide bearing amine groups is introduced into the tube and left to react at a temperature of 50° C. for 17 hours under argon and with magnetic stirring (septum). The slide is then rinsed with acetone, and then dried with nitrogen blowing.

The residual amine groups are then destroyed by amidation with trifluoroacetic anhydride according to the following protocol: 30 ml of THF dried on a molecular sieve are introduced into a tube, followed by 1 ml of trifluoroacetic anhydride. The slide from the preceding step is then introduced and left to react for 2.5 minutes under argon with magnetic stirring, at ambient temperature. The slide is removed and then rinsed by dipping in deionized water for 5 minutes, and then with jets of deionized water and, finally, dried by nitrogen blowing.

The slide is analyzed by IRRAS before and after each of the steps; the spectra thus obtained are given in FIG. 13 in the appendix.

Before the amidation reaction, the IRRAS analysis reveals the carboxylic acid group C═O elongation bands (1700 cm⁻¹), and also the amide II bands at 1591 cm⁻¹, which pleads in favor of a structure that is at least partially functionalized, and has the structure: R—(CH₂—NH₂)ₓ—(CH₂—NH(C═O)—(CH₂)₃—COOH)_{y}, where y/(x+y) is the degree of substitution of the initial amine groups with the glutaric anhydride, and R is the backbone of the electrografted PMAN.

After the amidation reaction, the IRRAS analysis confirms the carboxylic acid group C═O elongation bands (1700 cm¹), and also the amide II bands at 1591 cm⁻¹, and reveals the CF₃ group C—F elongation bands (1203 cm⁻¹), pleading in favor of a functionalized structure having the following structure:
R—(CH₂—NH(C═O)CF₃)ₓ— (CH₂—NH(C═O)— (CH₂)₃—COOH)_{y}
in which y/(x+y) is the degree of substitution of the initial amine groups by the glutaric anhydride, and R is the backbone of the electrografted PMAN.

The surface thus functionalized is then reacted with a 15 μM solution of the ODN (15-mer) aminated in the 5′ position, in deionized water, in the presence of N-hydroxysuccinimide (NHS) and of 1,3-(dimethylaminopropyl)-3-ethylcarbodiimide hydrochloride (EDC) at ambient temperature for 15 hours.

The slide is removed, rinsed with deionized water, and dried by nitrogen blowing, and then analyzed by IRRAS and XPS.

The slide thus obtained is then reacted with a solution of the ODN strand complementary to the first strand attached, in deionized water, for 15 hours at ambient temperature, removed, rinsed with deionized water, and then dried by nitrogen blowing.

The IRRAS spectra thus obtained are given in FIG. 14 in the appendix.

The XPS spectra are given in FIG. 15 in the appendix.

Analysis of the IRRAS spectra before reaction with the ODN strand complementary to the first strand attached reveals the appearance of nitrogenous base amide bands and also of the phosphate group P═O bond elongation bands, at around 1273 cm⁻¹.

The XPS analysis of the slide reveals the presence of phosphorus with a bond energy characteristic of DNA phosphate groups.

Analysis of the IRRAS spectra after reaction with the ODN strand complementary to the first strand attached confirms the nitrogenous base amide bands, and shows a significant increase in the intensity of the phosphate group P═O bond elongation bands, at around 1273 cm⁻¹. This observation is confirmed by the XPS analysis.

### EXAMPLE 12

### Electrografting of a Precursor Monomer Bearing a Spacer Arm

This example illustrates the electrografting of a monomer bearing a spacer arm comprising 6 carbon atoms, and bearing an amine group of interest (in the form of ammonium chloride): aminohexylmethacrylamide (AHMAA) of formula below:

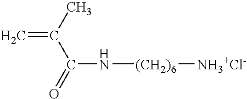

This example illustrates the possibility of electrografting a monomer bearing protic groups, a spacer arm, giving rise to an electrografted polymer in which the functional groups of interest are all accessible. They constitute an alternative pathway to that of Example No. 3 for obtaining an electrografted film bearing amine groups. Examples 4, 5, 6 and 11, repeated with the films of the present example, give similar results.

A poly-AHMAA (PAHMMA) film is produced on gold by carrying out 20 voltametric scans from −0.5 to −2.3 V/(Ag⁺/Ag) at 100 mV/s on a gold surface immersed in a 0.25 mol/l solution of AHMAA in DMF, in the presence of 5×10⁻² mol/l of TEAP. The slide is removed from the electrochemical cell and then vigorously rinsed with deionized water and then with acetone and, finally, dried under a stream of nitrogen.

Its IRRAS spectrum (not represented) is characteristic of the expected polymer, with in particular the characteristic bands of the ammonium group at 1613 and 1522 and the harmonic at 2050 cm⁻¹, and also a set of fine bands between 2400 and 2800 cm⁻¹, and the N—H⁺ elongation band at 3327 cm⁻¹, in addition to the amide bands at 1535 and 1465 cm⁻¹.

The PAHMAA film obtained is then dipped, with stirring, for 15 minutes, in a 1 mol/l sodium hydroxide (NaOH) solution. The slide is then rinsed with deionized water and then with acetone and, finally, dried as above. Its IRRAS spectrum (not represented) reveals the complete disappearance of the bands characteristic of ammonium groups, and the appearance of the bands characteristic of amine groups at 2933 cm⁻¹ (CH₂—NH₂ elongation) and 3360 cm⁻¹ (primary amine N—H elongation). This result demonstrates the complete accessibility of the ammonium groups which are converted to amines by acid-base reaction with the sodium hydroxide.

The slide is then again dipped in a 1 mol/l hydrochloric acid solution for 20 minutes, and then rinsed and dried.

Its IRRAS spectrum (not represented) is, in all respects, identical to that obtained above, which shows that the amine groups formed are themselves completely converted, once again, to ammonium groups.

These results are confirmed by XPS (not represented), in which the presence of chlorine on the overall spectrum is clearly observed when the film is in the form of ammonium chloride, and its absence is observed when it is in amine form. At the same time, the region of the K threshold of the nitrogen (N1s) comprises two peaks at 400 (amide) and 402 eV (ammonium) when the film is in ammonium form, and a single peak centered at around 400.5 eV when it is in amine form.

### EXAMPLE 13

### Preparation of an Electrografted Dextran/Methacrylate Film

The aim of this example is to demonstrate that it is possible to electrograft a macromolecule partially derivatized with activated vinyl groups, and to have nonderivatized functional groups of said molecule for subsequent post-functionalization. The macromolecule used is a dextran functionalized with glycidyl methacrylate (GMA) groups.

The macroelectrophile considered, called dextran-GMA, is represented by the formula below:

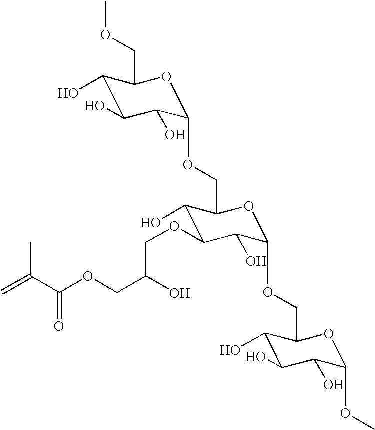

In the above formula, and in the interests of clarity, only one hydroxyl has been indicated as substituted with GMA. The proportion in fact varies according to the conditions of synthesis.

The dextran-GMA is obtained from a dextran of mass M=15000 and from glycidyl methacrylate (2,3-epoxypropyl methyl propenoate), according to the protocol described in W.N.E. by van Dijk-Wolthuis et al., Macromolecules, 1995, 28, 6317.

Analysis of the product by ¹H NMR and ¹³C NMR (not represented) shows that the dextran-GMA is obtained with a 77% degree of substitution.

A solution, called dextran-GMA solution, is prepared by dissolving 0.25 g of the dextran-GMA in 50 ml of DMF at 10⁻² mol/l in TEAP. The solution is therefore approximately at 3.3×10⁻⁴ mol/l of dextran-GMA.

Some gold surfaces, called gold slides, are prepared by spraying gold, by means of the Joule effect, onto glass slides pretreated with a chromium mist.

According to the process in accordance with the invention, the dextran-GMA is electrografted onto a gold slide used as a working electrode in a three-electrode assembly, in the dextran-GMA solution, according to the following potential protocol: voltametric conditions with 15 scans of Eᵢₙᵢₜᵢₐₗ=−0.6 V/(Ag⁺/Ag) to E_{final}=−2.8 V(Ag⁺/Ag) at a rate of −100 mV/s.

After rinsing of the slide with acetone and with water, a film 200 nm thick is obtained, the characteristics of which, verified by IR spectrophotometry, correspond to those of the poly(dextran-GMA) (FIG. 16 in which the bottom curve represents the IR spectrum of the slide before rinsing and the top curve represents the IR spectrum after rinsing).

The presence of a band at around 3500 cm⁻¹, characteristic of the numerous OH groups of the electrografted dextran, is in particular observed.

1. A solid support comprising at least one electrically conducting and/or semiconducting region containing a reducible oxide on its surface, characterized in that at least one zone of said surface is functionalized with an electrografted organic film obtained from electroactive organic precursors each comprising at least one functional group of interest, optionally in a mixture with electroactive organic precursors not comprising a functional group of interest, and in that the number of functional groups of interest accessible for the formation of a covalent, ionic or hydrogen bond with a complementary group within said film represents at least 90% of the total number of functional organic groups of interest.

2. The support as claimed in claim 1, characterized in that the organic precursors are chosen from:
polymerizable and electrograftable monomers bearing at least one organic functional group of interest;
polymerizable and electrograftable monomers bearing at least one functional group making it possible to obtain, by derivatization, the desired reactive functional organic group of interest;
molecules, macromolecules and objects functionalized with monomers bearing at least one organic functional group of interest or with monomers bearing at least one functional group making it possible to obtain, by derivatization, the desired reactive functional organic group of interest.

3. The support as claimed in claim 2, characterized in that the polymerizable monomers are chosen from activated vinyl monomers and molecules that are cleavable by nucleophilic attack, corresponding respectively to formulae (I) and (II) below: 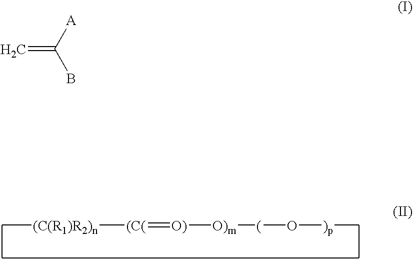
in which:
A, B, R₁ and R₂, which may be identical or different, represent a hydrogen atom, a C₁-C₄ alkyl radical, a nitrile radical or an organic function chosen from the following functions: hydroxyl, amine: —NHₓ with x=1 or 3, thiol, carboxylic acid, ester, amide: —C(═O)NH_{y} in which y=1 or 2, imide, imidoester, aromatic, acid halide: —C(═O)X in which X represents a halogen atom chosen from fluorine, chlorine or bromine, acid anhydride: —C(═O)OC(═O), nitrile, succinimide, phthalimide, isocyanate, epoxide, siloxane: —Si(OH)_{z} in which z is an integer between 1 and 3 inclusive, benzoquinone, carbonyldiimidazole, para-toluenesulfonyl, para-nitrophenyl chloroformate, ethylene and vinyl, or an organic group (or spacer arm) bearing at least one of the functions listed above; it being understood that at least one of A and B and that at least one of R₁ and R₂ represents one of said organic functions or an organic group bearing at least one of said functions;
n, m and p, which may be identical or different, are integers between 0 and 20 inclusive.

4. The support as claimed in claim 3, characterized in that the activated vinyl monomers of formula (I) are chosen from methacryloyl succinimide, hydroxyethyl methacrylate, methacrylonitrile, acrylonitrile, glycidyl acrylate and glycidyl methacrylate, acrylic acid, methacrylic acid, aminopropylmethacrylamide, aminohexylmethacrylamide, methacryloyl succinimide, acryloyl succinimide, methyl methacrylate, ethyl methacrylate, propyl methacrylate, butyl methacrylate, methyl cyanomethacrylate, methyl cyanoacrylate, 2- and 4-vinylpyridine and 4-chlorostyrene.

5. The support as claimed in claim 3, characterized in that the molecules that are cleavable by nucleophilic attack, of formula (II), are chosen from ethylene oxide, substituted ethylene oxides, butyrolactone, caprolactones and in particular ε-caprolactone.

6. The support as claimed in claim 2, characterized in that the molecules, macromolecules and objects functionalized with monomers are chosen from oligonucleotides, nucleic acid molecules, oligopeptides, polypeptides, proteins, oligosaccharides, polymers, fullerens, functionalized carbon nanotubes, and cells; said molecules, macromolecules and said objects being derivatized, totally or partially, with monomers corresponding to formulae (I) or (II).

7. The support as claimed in claim 1, characterized in that the electrically conducting or semiconducting surface is a stainless steel, steel, iron, copper, nickel, cobalt, niobium, aluminum, silver, titanium, silicon, titanium nitride, tungsten nitride or tantalum nitride surface, or a noble metal surface chosen from gold, platinum, iridium or platinum-iridium alloy surfaces.

8. The support as claimed in claim 1, characterized in that the density of the accessible functional groups of interest is preferably between 10⁴/μm² and 10¹⁰/μm².

9. A process for preparing a support comprising carrying out, in a single step, the electrografting of electroactive organic precursors onto at least one zone of at least one electrically conducting and/or semiconducting region containing a reducible oxide on its surface, of a solid support, by electrolysis, in an organic medium, of a composition containing, in said organic medium, at least one electroactive organic precursor comprising at least one functional group of interest, by bringing said composition into contact with said zone, the latter being subjected to a potential protocol during which it is brought, for all or part of the potential protocol, to a potential greater than or equal to a threshold electrical potential determined relative to a reference electrode, said threshold electrical potential being the potential beyond which the grafting of said precursors occurs, and in that a degree of accessibility of functional groups of interest of at least 90% (by number) is obtained:
a) by adjusting the potential protocol so as to produce a degree of grafting of less than or equal to 60%, and/or
b) by using a composition in which the functionalized electroactive organic precursors are present in a mixture with electroactive organic precursors not comprising a functional group of interest, the latter then representing from 0.1 to 95% of the total number of precursors present in said composition, and/or
c) by using electroactive organic precursors chosen from those in which the functional group of interest is borne at the end of a spacer arm.

10. The process as claimed in claim 9, characterized in that the degree of grafting is adjusted to a value of between 10 and 40%.

11. The process as claimed in claim 9, characterized in that, according to variant b), the electroactive organic precursors not comprising a functional group of interest represent from 0.1 to 50% of the total number of precursors present in said composition.

12. The process as claimed in claim 9, characterized in that the concentration of electroactive organic precursors comprising a functional group of interest is between 0.1 and 10 mol/l.

13. The process as claimed in claim 9, characterized in that the concentration of electroactive organic precursors not comprising a functional group of interest in said organic composition is between 10⁻³ and 18 mol/l.

14. The process as claimed in claim 9, characterized in that the electrolysis is carried out under voltametric conditions.

15. The process as claimed in claim 9, characterized in that the organic medium is chosen from dimethylformamide, ethyl acetate, acetonitrile and tetrahydrofuran.

16. The process as claimed in claim 9, characterized in that the organic medium also contains at least one support electrolyte.

17. An adhesion primer for attaching molecules of interest or objects bearing a complementary function comprising a solid support as defined in claim 1.

18. The adhesion primer as claimed in claim 17, characterized in that the molecules of interest are proteins and in that said support is then used as a bioactive surface or as a protein chip.

19. The adhesion primer as claimed in claim 17, characterized in that the molecules of interest are nucleic acid molecules and in that said support is then used as a bioactive surface or as a nucleic acid chip.

20. The adhesion primer as claimed in claim 17, characterized in that the molecules of interest or the objects bearing a complementary function are chosen from biocompatible polymers and in that said support is then used as a biocompatible surface or as a surface with encapsulating properties.

21. A method for bonding objects to conducting or semiconducting surfaces by means of surface chemical reactions, said method comprising using a solid support as defined in claim 1.
